(19) United States
(12) Patent Application Publication
(10) Pub. No.: US 2026/0148394 A1
(43) Pub. Date: May 28, 2026

(54) SYSTEM AND METHOD FOR DETERMINING A PROBABILITY OF REGISTERING IMAGES

(71) Applicant: Mazor Robotics Ltd., Caesarea (IL)

(72) Inventor: Dany Junio, Brookline, MA (US)

(21) Appl. No.: 19/150,043

(22) PCT Filed: Apr. 5, 2024
(86) PCT No.: PCT/IL2024/050352

§ 371 Date: Jul. 22, 2025

Related U.S. Application Data
(60) Provisional application No. 63/494,521, filed on Apr. 6, 2023.

(51) Int. Cl. G06T 7/32; A61B 6/12; A61B 34/20; G06T 7/33

(52) U.S. Cl. CPC G06T 7/32; A61B 6/12; A61B 34/20; G06T 7/337; A61B 2034/2051; A61B 2034/2065; G06T 2207/10116; G06T 2207/20076; G06T 2207/20081; G06T 2207/20084; G06T 2207/30012; G06T 2207/30052; G06T 2207/30204

(57) ABSTRACT

A method and system is disclosed for acquiring image data of a subject. The image data can be collected with an imaging system using various selection techniques. The selection techniques may be used to assist in generating selected images for viewing. WO

### FIELD

The present disclosure relates to imaging a subject, and particularly to a system to acquire image data registering to a pre-acquired image data.

### BACKGROUND

This section provides background information related to the present disclosure which is not necessarily prior art.

A subject, such as a human patient, may select or be required to undergo a surgical procedure to correct or augment an anatomy of the subject. The augmentation of the anatomy can include various procedures, such as movement or augmentation of bone, insertion of an implant (i.e., an implantable device), or other appropriate procedures. A surgeon can perform the procedure on the subject with images of the subject that can be acquired using imaging systems such as a magnetic resonance imaging (MRI) system, computed tomography (CT) system, fluoroscopy (e.g. C-Arm imaging systems), or other appropriate imaging systems.

Images of a subject can assist a surgeon in performing a procedure including planning the procedure and performing the procedure. A surgeon may select a two dimensional image or a three dimensional image representation of the subject. The images can assist the surgeon in performing a procedure with a less invasive technique by allowing the surgeon to view the anatomy of the subject without removing the overlying tissue (including dermal and muscular tissue) when performing a procedure.

### SUMMARY

This section provides a general summary of the disclosure, and is not a comprehensive disclosure of its full scope or all of its features.

Disclosed is an imaging system that is operable to acquire one or more image projections of a subject. In various embodiments, the image projections may be acquired and used to reconstruct an image of the subject. In various embodiments, as an alternative and/or in addition thereto, the projections may be viewed directly. The imaging system may include any selected imaging system, such as an x-ray imaging system. Accordingly, in various embodiments, the imaging system may generate a selected energy that is transmitted to and through the subject and is detected by a detector. Accordingly, the emitted energy may be transmitted through one or more filters prior to impinging or reaching the subject.

Image data may be acquired of the subject at any appropriate time. For example, first or pre-acquired image data may be image data that may be acquired first or prior to an action, such as acquired prior to performing a portion of the procedure. Current or second image data may be image data acquired after the first image data including after a portion of the procedure. In various embodiments, it may be selected to register the pre-acquired or first image data to the second image data. Registering the first and second image data may include defining a translation map between the first and second image data such that identical points in each of the image data are correlated.

The correlated image data allows for evaluating the second image data relative to the first image data based upon positions in the second image data and the first image data. In various embodiments, for example, a procedure may be planned using the first image data. The second image data may be acquired to determine whether the plan has been achieved. Registration of the second image data to the first image data, therefore, may assist in this determination. The second image data, however, may be acquired in an operating theater and after a portion of a procedure. Therefore, it may be selected to attempt to capture image data in as short a time as possible that is able to be registered to the first image data. Therefore, a system may be provided to analyze the image data substantially immediately after acquisition and prior to an attempt to perform a registration to determine whether a registration is likely or possible based upon an analysis of at least of the second image data.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of an imaging system in an operating theatre;

FIG. 2 is a detailed schematic view of an imaging system with a dual energy source system;

FIG. 3 is a flowchart of a method of determining a probability of registering a first image data to a second image data, according to various embodiments;

FIG. 4 is an exemplary illustration of a comparison of first image data to a second image data, according to various embodiments; and

FIG. 5 is an exemplary illustration of a comparison of first image data to a second image data, according to various embodiments.

Corresponding reference numerals indicate corresponding parts throughout the several views of the drawings.

### DETAILED DESCRIPTION

Example embodiments will now be described more fully with reference to the accompanying drawings.

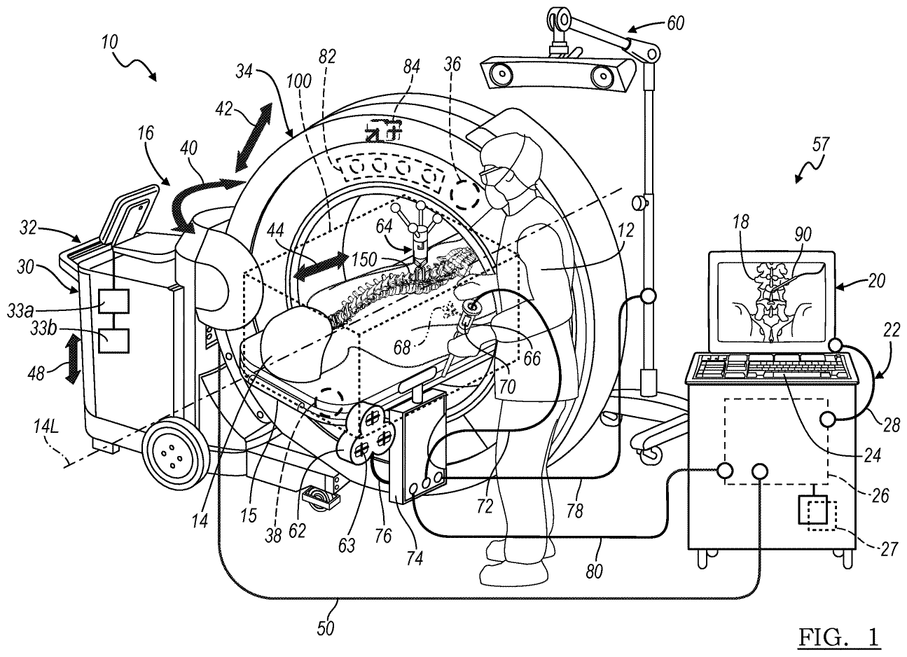

With reference to FIG. 1, in an operating theatre or operating room 10, a user, such as a surgeon 12, may perform a procedure on a subject, such as a patient, 14. In performing the procedure, the user 12 can use an imaging system 16 to acquire image data of the patient 14 to allow a selected system to generate or create images to assist in performing a procedure. The image data may be generated by x-ray powered for generating one or more projections of the subject 14. It is understood, however, that various types of image data may be collected and that the various types of image data may be used to generate or reconstruct an image 18. Further, the image data 18 generated with the imaging system 16 may be registered to a second image data set, such as a pre-acquired image. Thus, the image data may need to be a selected quality and/or amount. A system and method, as disclosed herein according to various embodiments, may allow for a determination of whether the image data will be able or likely to be registered to the second image data set.

The image 18 may include a model (such as a three-dimensional (3D) image) that can be generated using the image data, such as the image data acquired with the imaging system 16, and displayed as the image 18 on a display device 20. The display device 20 can be part of and/or connected to a processor system 22 that includes an input device 24, such as a keyboard, and a processor 26 which can include one or more processors or microprocessors incorporated with the processing system 22. The processing system 22 may further include selected types of non-transitory and/or transitory memory 27. A connection 28 can be provided between the processor 26 and the display device 20 for data communication to allow driving the display device 20 to display or illustrate the image 18.

The imaging system 16 may have various portions, such as those of an O-Arm® imaging system sold by Medtronic Navigation, Inc. having a place of business in Louisville, CO, USA. The imaging system 16 may also include and/or alternatively include various portions such as those disclosed in U.S. Patent App. Pubs. 2012/0250822, 2012/0099772, and 2010/0290690, all incorporated herein by reference. The imaging system 16, however, may be any appropriate imaging system such as a C-arm imaging system, a fluoroscopic imaging system, magnetic resonance imager (MRI), computer tomography (CT), etc.

The imaging system 16 may include a mobile cart 30 to allow the imaging system 16 to be mobile. The imagining system 16 may further include a controller and/or control system 32. The control system 32, in various embodiments, may be incorporated into the cart 30 or other appropriate location. Further, the control system 32 may include a processor 33a and a memory 33b (e.g., a non-transitory memory). The memory 33b may include various instructions that are executed by the processor 33a to control the imaging system, including various portions of the imaging system 16.

An imaging gantry 34 of the imaging system 16 may have positioned therein a source unit or system 36 and a detector 38 and may be connected to the mobile cart 30. The gantry 34 may be O-shaped or toroid shaped, wherein the gantry 34 is substantially annular and includes walls that form a volume in which the source unit 36 and detector 38 may move. The mobile cart 30 can be moved from one operating theater to another. The gantry 34 can move relative to the cart 30, as discussed further herein. This allows the imaging system 16 to be mobile and moveable relative to the subject 14 thus allowing it to be used in multiple locations and with multiple procedures without requiring a capital expenditure or space dedicated to a fixed imaging system. The processor(s) 26, 33a may include a general purpose processor or a specific application processor and the memory system(s) 27, 33b nay be a non-transitory memory such as a spinning disk or solid state non-volatile memory and/or transitory, and or remote having a connection to the processor. For example, the memory system may include instructions to be executed by the processor to perform functions and determine results, as discussed herein.

The source unit 36 may be an x-ray source, also referred to as an emitter, that can emit x-rays toward and/or through the patient 14 to be detected by the detector 38. As is understood by one skilled in the art, the x-rays emitted by the source 36 can be emitted in a cone and detected by the detector 38. The source/detector unit 36/38 is generally diametrically opposed within the gantry 34. The detector 38 can move in a 360° motion around the patient 14 within the gantry 34 with the source 36 remaining generally 180° opposed (such as with a fixed inner gantry or moving system also referred to as a rotor) to the detector 38.

The gantry 34 can move isometrically relative to the subject 14, which can be placed on a patient support or table 15, generally in the direction of arrow 40 as illustrated in FIG. 1. The gantry 34 can also tilt relative to the patient 14 illustrated by arrows 42, move longitudinally along the line 44 relative to a longitudinal axis 14L of the patient 14 and the cart 30, can move up and down generally along the line 46 relative to the cart 30 and transversely to the patient 14, to allow for positioning of the source/detector 36/38 relative to the patient 14. The imaging device 16 can be precisely controlled to move the source/detector 36/38 relative to the patient 14 to generate precise image data of the patient 14. The imaging device 16 can be connected with the processor 26 via connection 50 which can include a wired or wireless connection or physical media transfer from the imaging system 16 to the processor 26. Thus, image data collected with the imaging system 16 can be transferred to the processing system 22 for navigation, display, reconstruction, etc.

The source 36, as discussed herein, may include one or more sources of x-rays for imaging the subject 14. In various embodiments, the source 36 may include a single source that may be powered by more than one power source to generate and/or emit x-rays at different energy characteristics. Further, more than one x-ray source may be the source 36 that may be powered to emit x-rays with differing energy characteristics at selected times.

According to various embodiments, the imaging system 16 can be used with an un-navigated or navigated procedure. In a navigated procedure a localizer and/or digitizer a navigation system 57, including either or both of an optical localizer 60 and/or an electromagnetic localizer 62, can be used to generate a field and/or receive and/or send a signal within a navigation domain relative to the patient 14. The navigated or navigational space or domain relative to the patient 14 can be registered to the image 18. Correlation, as understood in the art, is to allow registration of a navigation space defined within the navigational domain and an image space defined by the image 18. A patient tracker or dynamic reference frame 64 can be connected to the patient 14 to allow for a dynamic registration and maintenance of registration of the patient 14 to the image 18.

The patient tracking device or dynamic registration device 64 and an instrument 66 can then be tracked relative to the patient 14 to allow for a navigated procedure. The instrument 66 can include a tracking device, such as an optical tracking device 68 and/or an electromagnetic tracking device 70, to allow for tracking of the instrument 66 with either or both of the optical localizer 60 and/or the electromagnetic localizer 62. The instrument 66 can include a communication line 72 with a navigation/probe interface device 74 such as the electromagnetic localizer 62 with communication line 76 and/or the optical localizer 60 with communication line 78. The interface 74 can then communicate with the processor 26 with a communication line 80. It will be understood that any of the communication lines 28, 50, 76, 78, or 80 can be wired, wireless, physical media transmission or movement, or any other appropriate communication. Nevertheless, the appropriate communication systems can be provided with the respective localizers to allow for tracking of the instrument 66 relative to the patient 14 to allow for illustration of a tracked location of the instrument 66 relative to the image 18 for performing a procedure.

One skilled in the art will understand that the instrument 66 may be any appropriate instrument, such as a ventricular or vascular stent, spinal implant, neurological stent or stimulator, ablation device, or the like. The instrument 66 can be an interventional instrument or can include or be an implantable device. Tracking the instrument 66 allows for viewing a pose (including x, y, z position and orientation) of the instrument 66 relative to the patient 14 with use of the registered image 18 without direct viewing of the instrument 66 within the patient 14.

Further, the gantry 34 can include a tracking device such as an optical tracking device 82 and/or an electromagnetic tracking device 84 to be tracked with the respective optical localizer 60 or electromagnetic localizer 62. Accordingly, the imaging device 16 can be tracked relative to the patient 14 as can the instrument 66 to allow for initial registration, automatic registration, or continued registration of the patient 14 relative to the image 18. Registration and navigated procedures are disclosed in U.S. Pat. No. 8,238,631, incorporated herein by reference. Upon registration and tracking of the instrument 66, an icon 90 may be displayed relative to, including superimposed on, the image 18. The image 18 may be an appropriate image and may include a long film image, 2D image, 3D image, or any appropriate image as discussed herein.

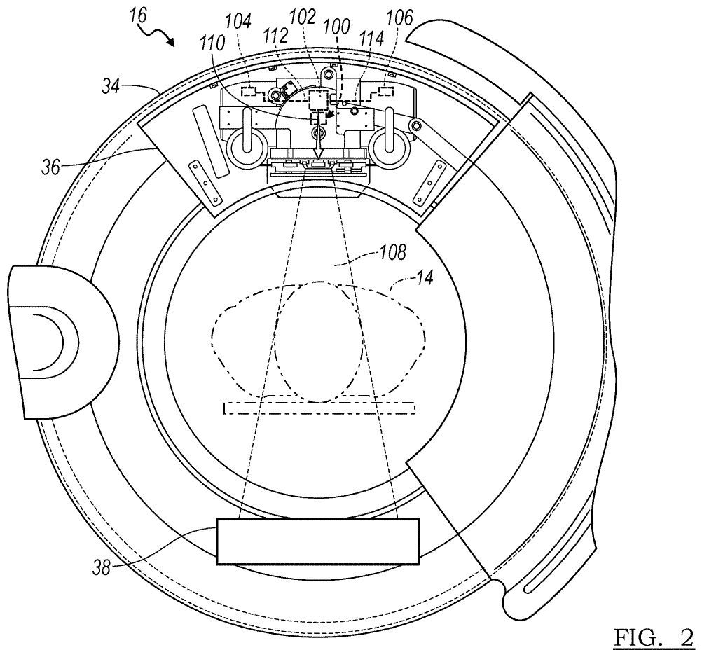

Turning reference to FIG. 2, according to various embodiments, the source unit 36 may include various components or features, as discussed herein. For example, the source unit 36 may include a x-ray source such as a single x-ray tube 100 that can be connected to a switch 102 that can interconnect a first power source A 104 and a second power source B 106 with the x-ray tube 100. X-rays can be emitted from the x-ray tube 100 generally in a cone shape 108 towards the detector 38 and generally in the direction from the source 100 as indicated by arrow, beam arrow, beam or vector 110. It is understood, however, that selected filters and/or adjustments may be made to alter the shape of the beam such that it is not a cone shape beam 108.

The switch 102 can switch between the power source A 104 and the power source B 106 to power the x-ray tube 100 at different voltages and/or amperages to emit x-rays at different energy characteristics generally in the direction of the vector 110 towards the detector 38. The vector 110 may be a central vector or ray within the cone 108 of x-rays. An x-ray beam may be emitted as the cone 108 or other appropriate geometry. The vector 110 may include a selected line or axis relevant for further interaction with the beam, such as with a filter member, as discussed further herein. Imaging systems and related filter members and collimator systems may be similar to those as disclosed in U.S. Pat. No. 10,682,103, incorporated herein by reference. In addition, various filter members and collimator systems along with various reconstruction and/or imaging techniques may be similar to those as disclosed in U.S. Pat. No. 10,881,371, incorporated herein by reference.

It will be understood, however, that the switch 102 can also be connected to a single variable power source that is able to provide power characteristics at different voltages and/or amperages rather than the switch 102 that connects to two different power sources A 104 and B 106. Also, the switch 102 can be a switch that operates to switch a single power source between different voltages and amperages. Further, the source unit 36 may include more than one source, such as x-ray sources, that are each configured or operable to emit x-rays at one or more energy characteristic and or different energy characteristic. The switch, or selected system, may operate to power the two or more x-rays tubes to generate x-rays at selected times.

Dual energy imaging systems may include those disclosed in U.S. Pat. App. Pub. No. 2012/0099768 and U.S. Pat. No. 9,769,912, both incorporated herein by reference.

To acquire a projection, also referred to as an image projection or generally as an image, the patient 14 can be positioned within the x-ray cone 108. Image data of the patient 14 is then acquired at the detector 38 based upon the emission of x-rays in the direction of vector 110 towards the detector 38. Generation of x-ray projections is may be used to collect or acquire image data of the subject for generation of images, as discussed herein.

It is understood, however, that the imaging system may also be used to generate projections with a single power. Thus, a single or dual power imaging system may be used to generate image data projections. The projections, regardless of how they are collected, may be used to generate images.

The images 18 may be generated by reconstruction from the image data. In various embodiments, an iterative or algebraic process can be used to reconstruct the image 18. The image 18 may include a model of at least a portion of the patient 14 based upon the acquired image data. It is understood that the model may include a three-dimensional (3D) rendering of the imaged portion of the patient 14 based on the image data. The rendering may be formed or generated based on selected techniques, such as those discussed herein.

The power sources can power the x-ray tube 100 to generate two dimension (2D) x-ray projections of the patient 14, selected portion of the patient 14, or any area, region or volume of interest. The 2D x-ray projections can be reconstructed, as discussed herein, to generate and/or display three-dimensional (3D) volumetric models of the patient 14, selected portion of the patient 14, or any area, region or volume of interest. As discussed herein, the 2D x-ray projections can be image data acquired with the imaging system 16, while the 3D volumetric models can be generated or model image data.

For reconstructing or forming a 3D volumetric image, appropriate algebraic techniques include Expectation maximization (EM), Ordered Subsets EM (OS-EM), Simultaneous Algebraic Reconstruction Technique (SART) and Total Variation Minimization (TVM), as generally understood by those skilled in the art. The application to perform a 3D volumetric reconstruction based on the 2D projections allows for efficient and complete volumetric reconstruction. Generally, an algebraic technique can include an iterative process to perform a reconstruction of the patient 14 for display as the image 18. For example, a pure or theoretical image data projection, such as those based on or generated from an atlas or stylized model of a “theoretical” patient, can be iteratively changed until the theoretical projection images match the acquired 2D projection image data of the patient 14. Then, the stylized model can be appropriately altered as the 3D volumetric reconstruction model of the acquired 2D projection image data of the selected patient 14 and can be used in a surgical intervention, such as navigation, diagnosis, or planning. The theoretical model can be associated with theoretical image data to construct the theoretical model. In this way, the model or the image data 18 can be built based upon image data acquired of the patient 14 with the imaging device 16.

The projection image data may be 2D projections and may be acquired by substantially total or partial annular or 360° orientation movement of the source/detector 36/38 around the patient 14 due to positioning of the source/detector 36/38 moving around the patient 14 in the optimal movement. An optimal movement may be a predetermined movement of the source/detector 36/38 in a circle alone or with movement of the gantry 34, as discussed above. An optimal movement may be one that allows for acquisition of enough image data to reconstruct a select quality of the image 18. This optimal movement may allow for minimizing or attempting to minimize exposure of the patient 14 and/or the user 12 to x-rays by moving the source/detector 36/38 along a path to acquire a selected amount of image data without more or substantially more x-ray exposure.

Also, due to movements of the gantry 34, the detector need never move in a pure circle, but rather can move in a spiral helix, or other rotary movement about or relative to the patient 14. Also, the path can be substantially non-symmetrical and/or non-linear based on movements of the imaging system 16, including the gantry 34 and the detector 38 together. In other words, the path need not be continuous in that the detector 38 and the gantry 34 can stop, move back the direction from which it just came (e.g. oscillate), etc. in following the optimal path. Thus, the detector 38 need never travel a full 360° around the patient 14 as the gantry 34 may tilt or otherwise move and the detector 38 may stop and move back in the direction it has already passed.

Image data acquired with the imaging system 16 and/or other image data may be used for a surgical navigation procedure using the system 57, as discussed further herein, may incorporate various portions or systems, such as those disclosed in U.S. Pat. Nos. RE 44,305; 7,697,972; 8,644,907; and 8,842,893; and U.S. Pat. App. Pub. No. 2004/0199072, all incorporated herein by reference. Various components or systems may be used in combination with or incorporated with the navigation system 57, such as the imaging system 16. It is understood, however, that the imaging system 16 may be used separate and independent of the navigation system 57.

The instrument 66 may be tracked in a trackable volume or a navigational volume by one or more tracking systems. Tracking systems may include one or more tracking systems that operate in an identical manner or more and/or different manner or mode. For example, the tracking system may include an electro-magnetic (EM) localizer 62, as illustrated in FIG. 1. In various embodiments, it is understood by one skilled in the art, that other appropriate tracking systems may be used including optical (including the optical or camera localizer 60), radar, ultrasonic, etc. The discussion herein of the EM localizer 62 and tracking system is merely exemplary of tracking systems operable with the navigation system 57. The position of the instrument 66 may be tracked in a tracking or navigation volume that is physical space generally defines relative to the subject 14. The tracked pose may be illustrated as a graphical representation or graphical overlay, also referred to as an icon 90 with the display device 20. In various embodiments, the icon 90 may be superimposed on the image 18 and/or adjacent to the image 18. As discussed herein, the navigation system 57 may incorporate the display device 20 and operate to render the image 18 from selected image data, display the image 18, determine the position of the instrument 66, determine the position of the icon 90, etc.

With continuing reference to FIG. 1, the EM localizer 62 is operable to generate electro-magnetic fields with an included transmitting coil array (TCA) that includes one or more transmitting conductive coils 63 which is incorporated into the localizer 62. The localizer 62 may include one or more coil groupings or arrays. In various embodiments, more than one group is included and each of the groupings may include three coils, also referred to as trios or triplets. The coils may be powered to generate or form an electro-magnetic field by driving current through the coils of the coil groupings. As the current is driven through the coils, the electro-magnetic fields generated will extend away from the localizer 62 and form a navigation domain or volume 100, such as encompassing all or a portion of a head, spinal vertebrae, or other appropriate portion. The coils may be powered through the TCA controller and/or power supply 74. It is understood, however, that more than one of the EM localizers 62 may be provided and each may be placed at different and selected locations.

The navigation domain or volume 100 generally defines a navigation space or patient space. As is generally understood in the art, the instrument 66, such as a drill, lead, implant (e.g. screw) etc., may be tracked in the navigation space that is defined by a navigation domain relative to a patient or subject 14 with an instrument tracking device 70. For example, the instrument 66 may be freely moveable, such as by the user 12, relative to a dynamic reference frame (DRF) or patient reference frame tracker 64 that is fixed relative to the subject 14. Both the tracking devices 70, 64 may include tracking portions that are tracking with appropriate tracking systems, such as sensing coils (e.g., conductive material formed or placed in a coil) that senses and are used to measure a magnetic field strength, optical reflectors, ultrasonic emitters, etc. Due to the instrument tracking device 70 connected or associated with the instrument 66, relative to the DRF 64, the navigation system 57 may be used to track the position of the instrument 66 relative to the DRF 64.

The navigation volume or patient space may be registered to an image space defined by the image 18 of the subject 14 and the icon 90 representing the instrument 66 may be illustrated at a navigated (e.g. determined) and tracked position with the display device 20, such as superimposed on the image 18. Registration of the patient space to the image space and determining a position of a tracking device, such as with the tracking device 70, relative to a DRF, such as the DRF 64, may be performed as generally known in the art, including as disclosed in U.S. Pat. Nos. RE 44,305; 7,697,972; 8,175,681; 8,503,745; 8,644,907; 8,737,708 and 8,842,893; 9,737,235; and U.S. Pat. App. Pub. No. 2004/0199072, all incorporated herein by reference and/or may also include the commercially available StealthStation® or Fusion™ surgical navigation systems sold by Medtronic Navigation, Inc. having a place of business in Louisville, CO.

Tracking information, including information regarding the electro-magnetic fields sensed with the tracking devices 70, 64 may be delivered via a communication system, such as the TCA controller 74, which also may be a tracking device controller, to the navigation processor system 22 including the navigation processor 26. Thus, the tracked position of the instrument 66 may be illustrated as the icon 90 relative to the image 18. Various other memory and processing systems may also be provided with and/or in communication with the processor system 26, including the memory system 27 that is in communication with the navigation processor 26 and/or the imaging processing unit 33a.

The tracking information may be used after registration to generate the icon 90. The registration can be performed as discussed herein, automatically, manually, or combinations thereof. Generally, registration allows a translation map to be generated of the physical location of the instrument 66 relative to the image space of the image data. The translation map allows the tracked position of the instrument 66 to be displayed on the display device 20 relative to the image data 18. The icon 90 can be used to illustrate the pose of the instrument 66 relative to the image data 18.

One or more fiducials may be identified on the subject 14. The fiducials may be anatomical (e.g., a spinous process) or artificial (e.g., an implant or connection). An artificial fiducial may be included with the DRF 64. In various embodiments, when the fiducial portions are imaged with the imaging device 16, or other appropriate imaging system, and image data is generated that includes or identifies the fiducial portions. The fiducial portions can be identified in image data automatically (e.g. with a processor executing a program such as by segmentation and/or identification of a selected shape), manually (e.g. by selection an identification by the user 12), or combinations thereof (e.g. by selection an identification by the user 12 of a seed point and segmentation by a processor executing a program). Methods of automatic imageable portion identification include those disclosed in U.S. Pat. No. 8,150,494 issued on Apr. 3, 2012, incorporated herein by reference. Manual identification can include selecting an element (e.g. pixel) or region in the image data wherein the imageable portion has been imaged. Regardless, the fiducial portions identified in the image data can be used as fiducial points or positions that can be used to register the image data or the image space of the image data with patient space.

In various embodiments, to register an image space or coordinate system to another space or coordinate system, such as a navigation space, the fiducial portions that are identified in the image 18 may then be identified in the subject space defined by the subject 14, in an appropriate manner. For example, the user 12 may move the instrument 66 relative to the subject 14 to touch the fiducial portions, if the fiducial portions are attached to the subject 14 in the same position during the acquisition of the image data to generate the image 18. It is understood that the fiducial portions, as discussed above in various embodiments, may be attached to the subject 14 and/or may include anatomical portions of the subject 14. Additionally, a tracking device may be incorporated into the fiducial portions and they may be maintained with the subject 14 after the image is acquired. In this case, the registration or the identification of the fiducial portions in a subject space may be made. Nevertheless, according to various embodiments, the user 12 may move the instrument 66 to touch the fiducial portions. The tracking system, such as with the optical localizer 60, may track the position of the instrument 66 due to the tracking device 68 attached thereto. This allows the user 12 to identify in the navigation space the locations of the fiducial portions that are identified in the Image 18. It is understood, however, that other appropriate systems and methods may be used to identify the feudal portions in or on the subject 14.

After identifying the positions of the fiducial portions in the navigation space, which may include a subject space, the translation map may be made between the subject space defined by the subject 14 in a navigation space and the image space defined by the image 18. Accordingly, identical or known locations allow for registration as discussed further herein.

During registration, the translation map is determined between the image data coordinate system of the image data such as the image 18 and the patient space defined by the patient 14. Once the registration occurs, the instrument 66 can be tracked with the tracking system that is registered to the image data to allow an identification and illustration of a position of the tracked instrument 66 as the icon 90 superimposed on the image 18. Registration of the image 18 (or any selected image data) to the subject 14 may occur at any appropriate time.

In various embodiments, the image base be based on image data that is a 2D image data that is generated with a cone beam. The cone beam that is used to generate the 2D image data may be part of an imaging system, such as the O-arm® imaging system. The 2D image data may then be used to reconstruct a 3D image or model of the imaged subject, such as the subject 14. The reconstructed 3D image and/or an image based on the 2D image data may be displayed. Thus, it is understood by one skilled in the art that the image 18 may be generated using the selected image data, such as from the imaging system 16.

In addition, the image 18 and or portions of the image data may be segmented, for various purposes, including those discussed further herein. In various embodiments, the segmentation, also referred to as the delineation, may be used to identify boundaries of various portions within the image 18, such as boundaries of one or more structures of the subject 14 and/or implants placed with the subject 14, such as the instrument 66. As discussed above, the instrument 66 may include an implant, such as a screw. A screw may include a screw such as a CD Horizon® Solara® Fenestrated Screws or a CD Horizon® Solara® Spinal System Screws, both sold by Medtronic, Inc. having a place of business in Minnesota, USA.

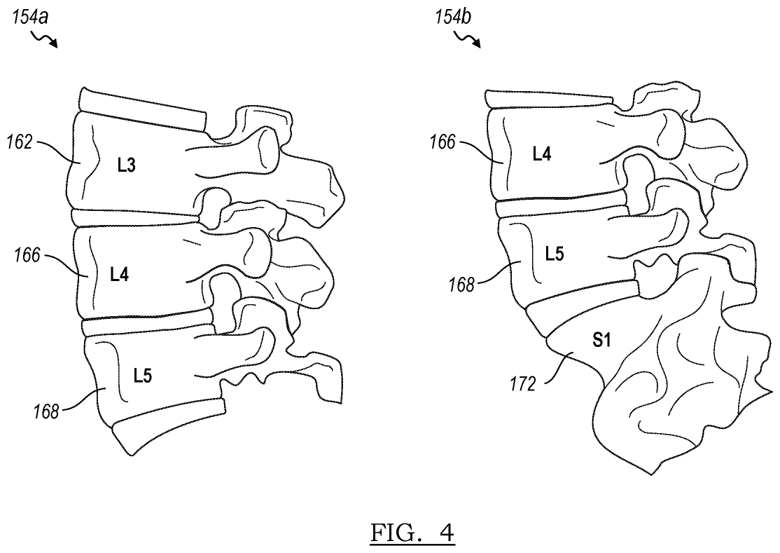

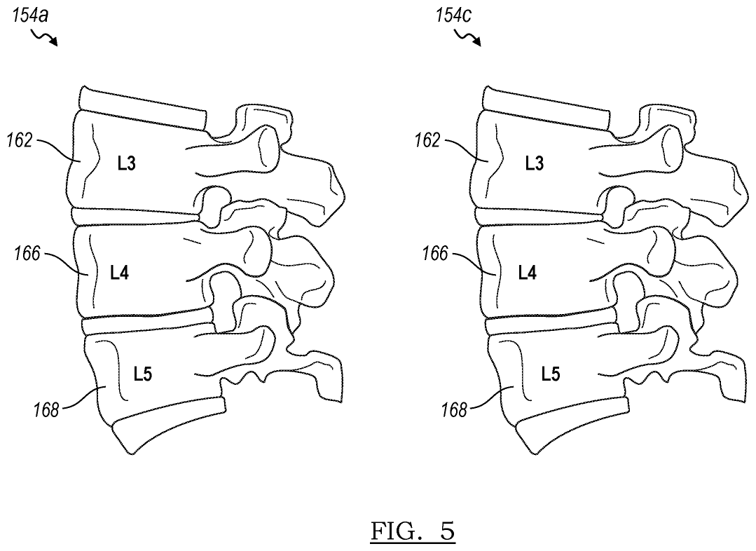

As discussed above, the subject 14 may be prepared for a procedure, such as an implantation, manipulation, or other appropriate procedure. In various embodiments, for example, a portion of a spine 150 may be manipulated and/or an implant may be position in the spine 150. An image of the spine may be generated in the image 18. The spine may include a plurality of vertebrae, which may be imaged such as in an image or included in an image data and rendered, as illustrated in FIGS. 4 and 5. For example, the spine 150 may be imaged and include image data 154. The image data may be used to generate images, such as the image 18, or other appropriate images. Further the spine may include various portions, such as vertebrae 158. The vertebrae may be specific vertebrae, such as a L3 vertebra 162, a L4 vertebra 166, and a L5 vertebra 168. Further additional portions of the spine 150 and/or other portions relative to the spine, such as a portion of the sacrum 172 may also be imaged and be rendered in the image 18. In addition, it is understood that other portions of the subject 14 may be imaged and the spine 150 is merely exemplary. Accordingly, the discussion herein regarding image data of the spine 150 and/or an image 18 of the spine based upon the image data acquired, is merely exemplary.

Further, as noted above, image data may be acquired of the patient 14 at any appropriate time. For example, in various embodiments, image data may be acquired of the patient or subject 14 prior to performing any portion of a procedure. Image data may be acquired with an appropriate image system, such as those discussed above including a CT and/or MRI scan. Image data may also include image data acquired with the imaging system 16, also at any appropriate time. Regardless, a first and second image data, as discussed further herein, may be acquired of the subject 14. The first and second image data may be acquired at different times, such as sequentially. In various embodiments, the first image data may be acquired at a time prior to a procedure and a second image data may be acquired after at least a portion of the procedure. It is further understood that during each acquisition of the first or second image data, various numbers of image data projections or portions may be acquired.

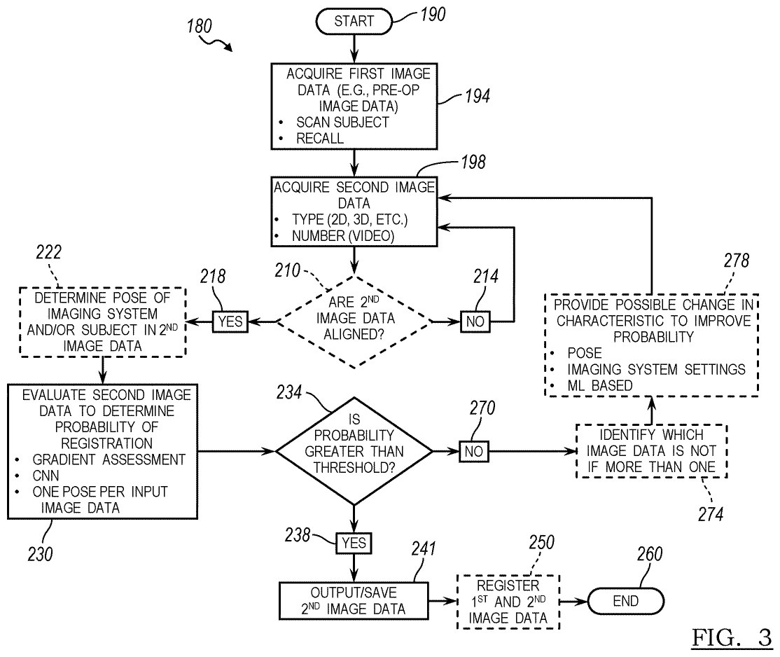

With initial reference to FIG. 3, a flowchart illustrating a process 180 is illustrated. The process 180 may be a process to assist in insuring or determining a probability that a second image data will register to a first image data. In various embodiments, for example, the first and second image data may be acquired of a similar or identical portion of the subject 14. However, if data is not acquired in a selected or appropriate manner in the second image data a registration to the first image data may not be possible. Accordingly, the process 180 may be used to assist in determining whether the second image data will likely be able to be registered to the first image data and/or if additional second image data should be acquired to ensure a registration. The registration allows a translation map to be made between the first and second image data. Thus, identical portions in both the first and second image data may be mapped to one another for comparison.

The process 180 may begin at start block 190. Starting the process 180 in start block 190 may include any appropriate process, such as initiating an acquisition of a second image data process. Accordingly, the process 180 may assist in determining whether the second image data is able to be registered to a first image data, as discussed further herein. The process 180, after starting at block 190 may include acquiring a first image data in block 194. Acquiring the first image data in block 194 may include scanning the subject 14. In various embodiments, for example, a procedure may include acquiring image data of the subject 14 with the imaging system 16. Acquiring image data in block 194 may also include recalling or accessing a memory and/or appropriate system including first image data. For example, the first image data may include a CT scan of the subject, a MRI scan of the subject, or any appropriate image data. Further the image data may be two-dimensional, three-dimensional, or include any appropriate dimensionality of the subject 14. Regardless, the first image data acquired in block 194 may be image data acquired prior to a second image data, as discussed further herein. According to various embodiments, the first image data may include image data acquired only before the second image data. For example, the acquired image data in block 194 may have been used to assist in performing a planning for a procedure on the subject 14.

Second image data may be acquired in block 198. As noted above, the second image data acquired in block 198 may be acquired or generate after the first image data acquired in block 194. The acquisition of the second image data may be acquired and/or made in any appropriate manner. For example, the imaging system 16 may be used to acquire image data of the subject 14 at a selected time. It is understood, however, that any appropriate imaging system may be used to acquire image data of the subject 14 such as a C-arm, a MRI imaging system, CT imaging system, or the like. In various embodiments, the second image data may be acquired after the first image data and/or after a selected procedure or portion of a procedure. For example, the first image data may be acquired of the subject 14 prior to the implantation of an implant, removal of a selected portion of the bone of the subject 14, or the like. The second image data may be acquired after implantation of an implant, removal of a bone portion, or the like.

The second image data may be any appropriate type of image data. For example, the imaging system 16 that may be the O-arm® imaging system and may acquire a 2D projection of the subject 14. In various embodiments, however, a three-dimensional image data may be acquired of the subject, such as with a CT scanner. The image data acquired of the subject 14 may be acquired, therefore, as any appropriate type.

According to various embodiments, the second image data acquired in block 198 may include a plurality of projections. For example, the second image data of the subject 14 may include a lateral image projection (e.g., medial to lateral (ML)) and/or an anterior-to-posterior (AP) projection of the subject 14. In various embodiments, the two projections may be used to generate or reconstruct a three-dimensional model or image of the subject 14 and therefore the two images may register to one another and/or to the first image data. Further, the imaging system 16 may be moved during the acquisition of a plurality of projections. For example, as illustrated FIG. 1, the source 84 may rotate around the subject 14, such as the long axis 14L of the subject 14, during the acquisition of image data. In various embodiments, the source 84 may move one to two degrees around the long axis 14L of the subject 14 and acquire a plurality of projections, such as two or more, during the movement and/or at selected positions in the range of movement. Therefore, each pose relative to the subject 14 may include the acquisition of a plurality of projections. For example, a plurality of projections may be acquired at both of the ML and the AP position relative to the subject 14.

Further, image data may be acquired of the subject 14 at more than only a ML and AP orientation. The imaging system 16 may move to acquire image data at oblique or other angles relative to the subject 14. Thus, image data acquired as the second image data in block 198 may be any appropriate image data acquired of the subject 14 for various purposes. Nevertheless, the second image data is generally selected to be registered to the first image data to assist in comparison between the first and second image data.

In registering the second image data to the first image data a comparison of the first image data to the second image data may be required. Therefore, selected image data information may be required in the second image data acquired in block 198 to ensure or allow for registration to the first image data acquired in block 194.

Turning reference to FIG. 4, for example, the first image data acquired in block 194 may be a first image data 154a. The second image data may be a second image data 154b. It may be selected to attempt to register the second image data 154b to the first image data 154a. Turning reference to FIG. 5, an alternative second image data 154c may also be acquired and it may also be selected to be registered to the first image data 15a. As discussed further herein, the two second image data 154b, 154c may be acquired sequentially and/or after determination that a first acquisition of the second image data is not appropriate or likely for registration to the first image data 154a. Briefly, for example, the first second image data 154b may include image data of the L4 vertebra 166, the L5 vertebra 168, and the S1 vertebra or sacrum 172. The second image data 154c may include image data of the L3 vertebra 162, the L4 vertebra 166, and the L5 vertebra 168. The first image data 154a may include image data of the L3 vertebra 162, the L4 vertebra 166, and the L5 vertebra 168. Accordingly, registration to the first image data 154a may require an appropriate amount of image data regarding the selected portions of the image in the first image data 154a which may not be present in the second image data 154b.

As discussed above, the second image data acquired in block 198 may be acquired as a single projection acquisition, multiple acquisitions, such as a ML and AP, and/or one or more moving acquisitions. The moving type acquisitions may include acquiring a plurality of projections over a small or selected movement of the imaging system 16, such as a one or two degree rotation thereof. This allows for the acquisition of the second image date.

After the acquisition, the second image data, a determination of whether the second image data is aligned may be made in block 210. If the image data is not aligned in block 210, a NO path 214 may be followed. It is understood that the determination of whether the image data is aligned in block 210 is optional and is not required for the process 180. The determination of whether the image date is aligned in block 210 may be whether or not the acquired second image data is aligned to itself and/or to a selected fiducial, such as a fiducial on a robot. For example, the Mazor® or Mazor X ® robot guidance systems may include a fiducial portion that is imaged in the second image data acquired in block 198. Each of the second image data acquisitions may be evaluated to determine that the image data is aligned or not. This may include a determination that the position of the fiducial in the image is the same between two images and to a reported position from the robot. Output regarding the alignment may be made to the user. A selection may be made to reacquire image data if the NOT path 210 is followed. However, if the image did is determined to be aligned in block 210 a YES path may be followed to determine a pose of the imaging system 16 and were the subject and the second image data in block 222.

A determination of whether the image date is aligned in block 210 is optional. Similarly, a determination or indication of the pose of the imaging system and/or the subject 14 is block 222 is also optional. The pose is a determination of the pose of the imaging system 16 to the subject 14 during the acquisition of the second image data. In various embodiments, a determination of the pose of the imaging system in block 222 may be based upon input of the user. Further, the imaging system 16 may be tracked relative to the subject, such as tracking the imaging system with the tracking device 82 and the subject with the tracking device 64. Therefore, a determination may be made with the navigation system 57 regarding a pose of the imaging system 16 relative to the subject 14.

A determination of the pose of the imaging system 16 may assist in evaluating the second image data relative to the first image data as discussed further herein. The determined pose may be input by the user, determined with the navigation system 16, determined with the imaging system 16, or other appropriate inputs. Nevertheless, the determined pose may include a selected position and/or orientation of the imaging system 16 relative to the subject 14 during the image data acquisition.

After the acquisition of the second image data in bock 198, at any appropriate time, such as after the optional portions of determining whether the second image data is misaligned and/or determining the pose of the imaging system, an evaluation of the second image data to determine a probability of registration to the first image data is made in block 230. The evaluation of the second image data to determine the probability of registration with the first image data may proceed in any appropriate manner. For example, as discussed above, the acquired second image data may include the acquisition of at least a first image data projection. The first image data projection may be compared and/or evaluated for a possibility or probability of a registration with the first image data. Similarly, and/or alternatively, the second image data may include a first projection and a second projection. The evaluation of a possibility or probability of registration may occur after the acquisition of both of the image data and/or after a single one of the projections. Further, as discussed above, the acquisition of the second image data may include an acquisition of a plurality of projections in a small area or volume, such as movement of the imaging system 16 in a range of movement such as 0.5°, 1°, 2°, or the like. The evaluation of a probability of registration of the second image data with the first image data in block 230 may include evaluating each of the projections in the movement in selecting if any of the projections has a selected probability for registration.

The evaluation of whether the second image data, or any projection thereof, has a probability of registration to the first image data allows for determining whether the registration may occur between the first and second image data prior to proceeding in a procedure on the subject 14. The determination of a probability of registration may include determining a percent likelihood of registration. The probability of registration, as discussed further herein, may then be compared to a threshold probability to determine whether the process 180 should proceed or iterate. According to various embodiments, the threshold percentage may be at least 30%, at least 40%, at least 50%, at least 60%, or any appropriate threshold. In various embodiments, the threshold may be any percentage that is greater than zero and/or any percentage that would ensure a certainty to achieve a registration.

Registration may allow for evaluation of a success of the selected planned procedure. The second image data may be selected to be registered to the first image data to ensure that the planned procedure has occurred for allowing of comparison of the second image data to the first image data. Additionally, the second image data may be registered to the first image data to assist in performing further portions of the procedure on the subject 14.

The determination of the probability of registration may allow for evaluating whether additional second image data may need to be or should be acquired and/or whether the procedure 180 may proceed. The evaluation of the second image data may occur according to any appropriate manner. Evaluation of the probability may be based on various features, such as edge detection, gradient determination, local distances, and sizes parameters (i.e., selected portions can be selected to be within a distance threshold of other portions including the scaling/rotation factors).

For example, a gradient assessment comparison may be made between the acquired second image data and the first image data. For example, the acquired second image data may include a two-dimensional projection which may have a gradient assessment comparison to a back projection through a three-dimensional model or image of the subject in the acquired first image data. Other appropriate gradient assessments may also be made to compare or evaluate a possibility of registration of the second image stated to the first image data.

In registration, the second image data may be registered to the first image data such that a translation map may be made between the first and second image data. The translation map may allow for the determination in the second image data of the same or similar portions in the first image data. For example, a translation map of the position of the portion of the vertebrae may be made. As noted above, a translation that may be made between the portions of the same or similar vertebrae of the subject 14, such as the L3 vertebrae 162.

Other probability evaluation processes or methods may include a convolutional neural network (CNN). A CNN may be any appropriate network, such as the CNN developed by Ronneberger et al. and initially described in “U-Net: Convolutional Networks for Biomedical Image Segmentation” at https://doi.org/10.1007/978-3-319-24574-4_28 and generally referred to as a U-net convolutional network. While the CNN is an exemplary machine learning system, any appropriate machine learning system may be used to determine a probability of registration of the second image data with the first image data. Machine learning systems, including the CNN, may be trained with image data to determine a probability of registration between the acquired second image data in block 198 and the acquired first image data from block 194. The determination of probability may allow for a probability that any portion or projection, such as a first protection, second projection, or any appropriate or selected rejection, may be registered to the acquired first image data from block 194.

In various embodiments, a network may be trained by being provided good and bad sets of image data that can or cannot be registered successfully to the original CT image, with the applicable annotation of good/bad images. The network would then use each layer to breakdown the image further and further to extract the parameters from both the original 2D and 3D images to find the correlations that make such a successful prediction. This may require breaking the image down to small pixel number patches.

The determined probability of registration in block 230 may be used to determine whether the probability is greater than a threshold in block 234. A threshold determination may be based on results of the training process of the machine learning discussed above. As discussed above, the probability may be determined for any one or more of projections that are acquired as a second image data from block 198. Thus, as more than one probability may be determined, such as one for each of a plurality of projections, a determination of whether the probability for each projection is greater than a threshold may be made in block 234.

The probability threshold in block 234 may be set for any appropriate value. For example, the threshold may be set at 10%, 20%, 30%, 50%, 60%, 70%, 90%, or any selected value. The probability may be set to allow for a fast acquisition of second image data, a high probability or near certainty of registration, or any other appropriate value. Therefore, the probability threshold may be predetermined and/or set by the user in any appropriate time. Nevertheless, if the probability determine determined in block 230 reaches or passes the threshold determined, received, or recalled in block 234, a YES path 238 may be followed.

The YES path 238 may then follow to the output or saving of the second image data in block 242. The outputting of the second image data may include displaying the second image data. Saving of the second image data may include saving the second for further analysis and/or use. Further the second image data may be registered to the first image data, optionally, in block 250. The registration of the first image data to the second image data may occur in any appropriate time and/or for the procedure. Therefore, the registration of the first image data and the second image stated in block 250 is not required in the process 180. Nevertheless, the process 180 may then END in block 260.

Thus, the process 180 may allow for an evaluation of whether the image data acquired in the second image data from block 198 has a selected threshold probability of registration with the first image data from block 194. If the probability is at or above a threshold and a registration may be likely this may allow for a procedure of the subject 14 to proceed efficiently. However, if the probability is determined to not be above the threshold, a NO path 270 may be followed.

The NO path 270 may allow for a determination of which image projection does not meet the probability threshold in block 274. As noted above, the acquired second image data may include a plurality of projections and, therefore, the identification of which projection does not meet the threshold may allow the user 12 to better identify which projection may need to be reacquired or acquired again as the second image data. In the process 180, therefore, multiple or alternative second image data may be acquired. The alternative second image data may be due to an iteration of the process 180 and/or to minimize a requirement for iterations of the process.

For example, the user may acquire one projection in the AP position relative to the subject 14 and the user may acquire a projection in the ML position relative to the subject 14. While a registration may not be possible based upon the acquisition of both projections, a registration may not be possible based upon only a single one of the projections. For example the imaging system 16, the subject 14, or other portions may have moved during only one of the projection acquisitions. An output and identification of which projection does not meet the probability threshold in block 274 may be made to the user 12.

In various embodiments, a provision of a possible change in characteristics to improve a probability may be made in block 278. The change in one or more characteristics may be various characteristics to acquire the second image data such as a pose of the imaging system 16, imaging system settings (e.g., power level, filter, etc.), and/or outputs from the machine learning system. For example, a change in characteristic of the imaging system 16 may include moving the imaging system a selected amount to achieve a better image acquisition of the subject 14 as the second image data and block 198. For example, as noted above, the second image data may be acquired after a portion of a procedure. Therefore, an implant may be positioned in the subject 14. The implant may cause distortion in the image data. Therefore, an output to change a characteristic may include a suggestion of a selected position to move the imaging system 16 to reduce artifacts in the acquired image data. Further, the imaging system may acquire image data at various different characteristics, as noted above including different powers, and a suggestion may include to change the acquisition to a different power, filter, beam pattern, etc. Regardless, the possible change in characteristics to improve a probability may be made and output in block 278. The characteristic change may be output in any appropriate manner, such as on the display of the imaging system 16, the display 20, or any other appropriate output.

The determination of the probability that is lower than a threshold in block 234 may be output in any appropriate manner to the user 12, such as a sound, a visual, a haptic feedback, or the like. Therefore, the user 12 may understand that a different or alternative second image data may need to be acquired in block 198. Following the NO path 270 may allow for the acquisition of the second image data in block 198. The acquisition of an alternative or additional second image data in block 198 may allow for the iteration of the process 180 to ensure that the image data acquired in block 198 has a selected probability of registration with the first image data. Thus, the process 180 may iterate until a selected probability threshold is reached in block 234. The process 180 may, therefore, END in block 216.

As noted above, the process 180 may allow for a determination or selection of whether the second image data has a probability of a threshold to be registered with the first image data in block 234 based upon the evaluation of block 230. Turning reference to FIG. 4 and FIG. 5, exemplary image data are illustrated. Initially, in FIG. 4, the first image data 154a may include selected vertebrae including the L3 vertebra 162, the L4 vertebra 166, and the L5 vertebra 168. It is understood that to the image data 154a may include any appropriate portions of the subject 14 and the exemplary of three vertebrae 162-168 are for the current discussion. Further, as noted above, the first image data 154a may include any appropriate type of image, such as a three-dimensional image, two-dimensional image, or the like.

During an acquisition of the second image data, the image data may acquire, however, only the vertebrae L4 166, L5 168, and S1 172. It may be determined that a probability of registration does not meet the threshold based upon an evaluation in block 230 given the second image data 154b. It is understood that the second image data 154b includes a lack of selected anatomical portions, that the second image data may also include a lack of clarity, lack of focus, lack of gradient differences, or the like that may be evaluated in block 230 to determine that the probability is less than a threshold in block 234.

Turning reference to FIG. 5, the first image data 154a may be substantially identical to the first image data 154a discussed above. The second image data 154c acquired of the subject 14 may include the same vertebrae 162 -168 of the subject 14 as in the first image data 154a. The second image data 154c may either be the first second acquired in block 198 or an alternative second image data acquired a block 198 after determination that a previous second image data acquisition does not reach the threshold in block 234. Nevertheless, the second image data 154c may include a selected type and/or amount of image data to allow for registration with the first image data 154a based upon the evaluation from block 230 and the probability is or is greater than the threshold in block 234. A registration may then occur in the procedure may and in block 260.

The second image data, therefore, may be acquired any appropriate number of times to achieve a selected probability of registration with the first image data. The process 180 may allow for the acquisition of the second image data during only a selected period of a procedure without requiring repositioning of the imaging system 16, acquisition of additional image data when not necessary, efficiency of a procedure on the subject 14, and the like. The process 180 may allow for assisting to ensure and/or increasing probability of registration of the acquired second image data with the first image data without attempting the registration process. In other words, the process 180 may assist in determining whether the acquired second image data may be registered with the first image data without attempting a registration process, according to any appropriate manner, and having a failure of the registration process.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a special purpose computer created by configuring a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C #, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor or module or ‘controller’ may be replaced with the term ‘circuit.’ The term ‘module’ may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A system to evaluate image data for use during a procedure, comprising:
a processor configured to:
evaluate a first image data;
determine a probability of registration of the second image data to the first image data;
determine whether the determined probability is greater than a selected probability threshold; and
output whether the selected probability threshold is reached.

2. The system of claim 1, wherein the processor is further configured to output a proposed change in imaging characteristics to at least reach the selected probability threshold by the second image data.

3. The system of claim 1, wherein the processor is further configured to determine whether the second image data is aligned to a selected portion.

4. The system of claim 1, wherein the determination of the probability of registration of the second image data to the first image data is based on a machine learning system.

5. The system of claim 4, wherein the machine learning system is a convolutional neural network.

6. The system of claim 1, wherein the second image data is acquired after the first image data;
wherein the first image data is evaluated relative to the second image data to determine the probability of registration.

7. The system of claim 1, further comprising:
an imaging system configured to acquire the second image data.

8. The system of claim 7, wherein the imaging system is a x-ray imaging system.

9. The system of claim 1, further comprising:
a navigation system including a tracking system configured to determine a pose of an instrument relative to a subject and determine a graphical illustration to represent the determined pose of the instrument.

10. A method to evaluate image data for use during a procedure, comprising:
evaluating a first image data relative to a second image data;
determining a probability of registration of the second image data to the first image data;
determining whether the determined probability is greater than a selected probability threshold;
outputting whether the selected probability threshold is reached.

11. The method of claim 10, further comprising:
outputting a proposed change in imaging characteristics to at least reach the selected probability threshold by the second image data.

12. The method of claim 10, wherein the determination of the probability of registration of the second image data to the first image data is based on a machine learning system.

13. The method of claim 10, further comprising:
acquiring the second image data with an imaging system.

14. The method of claim 10, further comprising:
executing instructions with a processor to at least determine the probability of registration of the second image data to the first image data and determined whether the determined probability is greater than the selected probability threshold.

15. The method of claim 10, further comprising:
navigating an instrument relative to a subject with a navigation system including a tracking system configured to determine a pose of the instrument relative to the subject; and
determining a graphical illustration to represent the determined pose of the instrument.

16. A system to evaluate image data for use during a procedure, comprising:
an imaging system configured to acquire a second image data of a subject;
a processor configured to:
evaluate a first image data relative to the second image data;
determine a probability of registration of the second image data to the first image data;
determine whether the determined probability is greater than a selected probability threshold;
output whether the selected probability threshold is reached;
wherein the registration of the first image data to the second image data includes a translation map of the first image data to the second image data.

17. The system of claim 16, wherein the processor is further configured to execute instructions to output whether the determined probability is greater than the selected probability threshold; and
if the determined probability is less than the selected probability threshold output a proposed change in imaging characteristics to have at least reach the selected probability threshold by the second image data.

18. The system of claim 16, wherein the determination of the probability of registration of the second image data to the first image data is a convolutional neural network.

19. The system of claim 16, further comprising:
a navigation system including a tracking system;
wherein the tracking system is configured to track a tracking device;
wherein the navigation system is configured to determine a pose of an instrument relative to the subject and determine a graphical illustration to represent the determined pose of the instrument.

20. The system of claim 19, further comprising:
a display device configured to display the first image data or the second image data and the graphical illustration to represent the determined pose of the instrument.
